(19) United States
(12) Patent Application Publication
(10) Pub. No.: US 2009/0076569 A1
(43) Pub. Date: Mar. 19, 2009

(54) FITTING A PROSTHETIC HEARING IMPLANT FOR A RECIPIENT

(75) Inventors: Peter A. Busby, Caulfield (AU); Jim Patrick, Roseville (AU)

Correspondence Address: Connolly Bove Lodge & Hutz LLP, Suite 1100, 1875 Eye Street, NW, Washington, DC, 20006, US

(73) Assignee: Cochlear Limited, Lane Cove (AU)

(21) Appl. No.: 12/181,098

(22) Filed: Jul. 28, 2008

Related U.S. Application Data
(63) Continuation of application No. 11/348,309, filed on Feb. 7, 2006.
(60) Provisional application No. 60/650,148, filed on Feb. 7, 2005.

(51) Int. Cl. A61F 11/04; A61N 1/36

(52) U.S. Cl. 607/57

(57) ABSTRACT

System and methods for fitting a cochlear implant for a recipient. In a fitting method, receiving, at an acoustic domain user interface, an acoustic target intensity for each of a plurality of frequency channels at which the recipient is to experience a desired percept; loading into a speech processor unit of the cochlear implant a MAP specifying a stimulation signal current level corresponding to a selected acoustic target; presenting the selected acoustic target to the cochlear implant so as to cause the cochlear implant to deliver electrical stimulation to the recipient at the current level corresponding to the selected acoustic target; and upon receipt of an external command: adjusting the current level corresponding to the selected acoustic target; and repeating the loading and the presenting steps.

### CROSS-REFERENCE TO RELATED APPLICATIONS

This application is a Continuation-In-Part of U.S. patent application Ser. No. 11/348,309, filed Feb. 7, 2006, which claims the benefit of U.S. Provisional Application No. 60/650,148, entitled “Electrically-stimulating hearing implant Programming Technique,” filed Feb. 7, 2005, all of which are hereby incorporated by reference herein.

### BACKGROUND

1. Field of the Invention

The present invention relates generally to prosthetic hearing implants, and more particularly, to fitting a prosthetic hearing implant for a recipient.

2. Related Art

There are many medical implants that deliver electrical stimulation to a patient or recipient (“recipient” herein) for a variety of therapeutic benefits. For example, electrically-stimulating prosthetic hearing implants such as auditory brain implants, or ABIs (also referred to as auditory brain stimulators) and Cochlear™ implants (also commonly referred to as Cochlear™ prostheses, Cochlear™ devices, Cochlear™ implant devices, and the like; generally and collectively referred to as “cochlear implants” herein), have been developed to provide a person having sensorineural hearing loss with the ability to perceive sound. The hair cells of the cochlea of a normal healthy ear convert acoustic signals into nerve impulses. People who are profoundly deaf due to the absence or destruction of cochlear hair cells are unable to derive suitable benefit from conventional hearing aid systems which amplify acoustic sound. Cochlear implants provide such persons with the ability to perceive sound by bypassing the hair cells of the cochlea and delivering an electrical stimulation to the cochlea, thereby inducing a hearing sensation.

Cochlear implants typically comprise external and implantable or internal components that cooperate with each other to provide sound sensations to a recipient. The external component includes a microphone that detects sound, such as speech and environmental sound, a speech processing unit that selects and converts detected sound, particularly speech, into a coded signal, a power source, such as a battery, and an external transmitter antenna.

The coded signal output by the speech processing unit is transmitted transcutaneously to an implanted receiver/stimulator unit commonly located within a recess of the temporal bone of the recipient. This transcutaneous transmission occurs via the external transmitter antenna which is positioned to communicate with an implanted receiver antenna typically disposed within the receiver/stimulator unit. This communication includes the coded sound signal and may also provide power to the implanted receiver/stimulator unit. Conventionally, this link has been in the form of a radio frequency (RF) link, although other communication and power links have been proposed and implemented.

The implanted receiver/stimulator unit also includes a stimulator that processes the coded signal and outputs electrical stimulation signals to an intra-cochlea electrode assembly. The electrode assembly typically has a plurality of electrodes mounted on a carrier member to apply electrical stimulation to the auditory nerve so as to produce a hearing sensation corresponding to the detected sound. The cochlea is tonotopically mapped; that is, partitioned into regions each responsive to stimulus signals in a particular frequency range. As such, each electrode or group of electrodes, referred to as a channel of the cochlear implant, of the implantable electrode assembly delivers a stimulation signal to a particular region of the cochlea. In the conversion of sound to electrical stimulation, frequencies are allocated to individual electrodes of the electrode assembly that lie in positions in the cochlea that are close to the region that would naturally be stimulated by such frequencies in a cochlea capable of normal hearing. This enables the cochlear implant to bypass the hair cells in the cochlea to directly deliver electrical stimulation to auditory nerve fibers, thereby allowing the brain to perceive hearing sensations resembling natural hearing sensations.

Auditory brain stimulators are used to treat a smaller number of recipients with bilateral degeneration of the auditory nerve. The auditory brain stimulator typically includes a planar electrode array which provides stimulation of the cochlear nucleus in the brainstem. A planar electrode array is one in which the electrode contacts are disposed on a two dimensional surface which is configured to be positioned proximal to the brainstem. Similar to cochlear implants, electrodes or groups of electrodes of the planar electrode array are sometimes referred to as channels of the auditory brain stimulator.

The effectiveness of a cochlear implant, auditory brain stimulator or other electrically-stimulating hearing implant is dependent not only on the device itself, but also on the success with which the device is configured for the recipient. Due to advances in electrically-stimulating hearing implant technology, configuring such devices, also referred to as “fitting,” “programming” or “mapping,” is a relatively complex process. Typically, a clinician, audiologist or other medical practitioner (generally and collectively referred to as “audiologist” herein) uses interactive software and computer hardware to create individualized programs, commands, data, settings, parameters, instructions, and/or other information (generally and collectively referred to as a “MAP” herein) that define the specific characteristics used to generate the electrical stimulation signals presented to the electrodes of the implanted electrode assembly.

Electrically-stimulating hearing implants offer a number of sophisticated MAP parameters that may be manipulated to improve sound quality and speech understanding. Today, most MAPs include at least two values for each frequency channel of the particular electrically-stimulating hearing implant. These values are referred to as the Threshold level (commonly referred to as the “THR” or “T-level;” “threshold level” herein) and the Maximum Comfortable Loudness level (commonly referred to as the Most Comfortable Loudness level, “MCL,” “M-level,” or “C;” simply “comfort level” herein). Threshold and comfort levels are psychophysical judgments of loudness that are measured in clinical units of electrical current, referred to as current units (cu). Threshold levels are comparable to acoustic threshold levels and indicate the current level at which a sound is barely audible. Comfort levels indicate the current level at which a sound is loud but comfortable.

Accurately determining MAP parameters requires specially trained audiologists having a detailed knowledge of how the electrical stimulation signals generated by the electrically-stimulating hearing implant are defined, generated and/or controlled. As such, audiologists lacking such knowledge are generally unable to effectively and efficiently perform the operations necessary to fit an electrically-stimulating hearing implant for a recipient.

### SUMMARY

In one aspect of the present invention, a method for fitting a cochlear implant for a recipient is provided. The method comprises: receiving, at an acoustic domain user interface, an acoustic target intensity for each of a plurality of frequency channels at which the recipient is to experience a desired percept; loading into a speech processor unit of the cochlear implant a MAP specifying a stimulation signal current level corresponding to a selected acoustic target; presenting the selected acoustic target to the cochlear implant so as to cause the cochlear implant to deliver electrical stimulation to the recipient at the current level corresponding to the selected acoustic target; and upon receipt of an external command: adjusting the current level corresponding to the selected acoustic target; and repeating the loading and the presenting steps.

In another aspect of the present invention a system for fitting a cochlear implant for a recipient is provided. The system comprises: means for receiving, at an acoustic domain user interface, an acoustic target intensity for each of a plurality of frequency channels at which the recipient is to experience a desired percept; means for loading into a speech processor unit of the cochlear implant a MAP specifying a stimulation signal current level corresponding to a selected acoustic target; means for presenting the selected acoustic target to the cochlear implant so as to cause the cochlear implant to deliver electrical stimulation to the recipient at the current level corresponding to the selected acoustic target; and upon receipt of an external command: means for adjusting the current level corresponding to the selected acoustic target; and means for repeating the loading and the presenting steps.

In a still other aspect of the present invention, a system for fitting a cochlear implant for a recipient is provided. The system comprises: an acoustic domain user interface configured to receive an acoustic target intensity for each of a plurality of frequency channels at which the recipient is to experience a desired percept; a MAP generator configured to load a MAP into a speech processor unit of the cochlear implant a MAP specifying a stimulation signal current level corresponding to a selected acoustic target; an acoustic signal generator configured to present the selected acoustic target to the cochlear implant so as to cause the cochlear implant to deliver electrical stimulation to the recipient at the current level corresponding to the selected acoustic target; and upon receipt of an external command, the system is configured to adjust the current level corresponding to the selected acoustic target, the MAP generator is configured to repeat the loading, and the signal generator is configured to repeat the presentation.

In a still other aspect of the present invention, a method for fitting for a recipient a cochlear implant using acoustic targets each having an acoustic intensity and frequency specified in an acoustic domain environment is provided. The method comprises: determining for each acoustic target the stimulating current level which will evoke a desired percept in the recipient.

### BRIEF DESCRIPTION OF THE DRAWINGS

Embodiments of the present invention are described herein in conjunction with the accompanying drawings, in which:

FIG. 1 is an exemplary electrically-stimulating hearing implant, namely a cochlear implant, which may be advantageously configured in accordance with embodiments of the present invention;

FIG. 2 is a schematic diagram of an exemplary fitting arrangement in which embodiments of a hearing implant fitting system is implemented to fit a cochlear implant for a recipient;

FIG. 3A is a high-level flow chart illustrating operations that may be performed while fitting an electrically-stimulating hearing implant utilizing an embodiment of the fitting system illustrated in FIG. 2;

FIG. 3B is a detail-level flow chart illustrating operations performed to determine stimulating current corresponding to acoustic targets in accordance with embodiments of the fitting operation of the present invention;

FIG. 4 is a high-level functional block diagram of one embodiment of the electrically-stimulating hearing implant fitting system illustrated in FIG. 2;

FIG. 5A is an exemplary target audiogram displayed to an audiologist by the fitting system illustrated in FIG. 2 to graphically present default acoustic targets in accordance with embodiments of the present invention;

FIG. 5B is the exemplary target audiogram illustrated in FIG. 5A subsequent to when an audiologist adjusts acoustic targets, in accordance with embodiments of the present invention;

FIG. 5C is the exemplary target audiogram illustrated in FIG. 5A subsequent to when an embodiment of the fitting system illustrated in FIG. 2 determines the current level corresponding to the threshold and comfort levels at two frequencies, in accordance with embodiments of the present invention;

FIG. 5D is the exemplary target audiogram illustrated in FIG. 5A during measurement of a threshold level with an embodiment of the fitting system illustrated in FIG. 2, in accordance with embodiments of the present invention; and

FIG. 6 illustrates the adjustment in current level of a measured comfort level, in accordance with embodiments of the present invention.

### DETAILED DESCRIPTION

Aspects of the present invention are generally directed to fitting an electrically-stimulating hearing implant. The fitting system provides an acoustic domain environment through which an audiologist specifies for a variety of frequency channels the intensity level at which a particular recipient is to experience a desired percept. For each of these acoustic targets the fitting system determines the stimulating current level that evokes the desired percept in the recipient. Specifically, embodiments of the fitting system repeatedly present each acoustic target to the implant and increment or decrement the corresponding current level until the recipient experiences the desired percept in response to the acoustic target. These current levels are included in the MAP stored in the implant for subsequent normal implant operations by that recipient. Because the acoustic targets are set by the audiologist in an acoustic domain environment, the audiologist is not required to have detailed knowledge of how electrical stimulation signals are defined, generated and/or controlled in order to perform the fit the electrically-stimulating hearing implant.

Electrically-stimulating hearing implants include but are not limited to auditory brain stimulators and cochlear implants. Cochlear implants use direct electrical stimulation of auditory nerve cells to bypass absent or defective hair cells that normally transduce acoustic vibrations into neural activity. Such devices generally use an electrode array inserted into the scala tympani of the cochlea so that the electrodes may differentially activate auditory neurons that normally encode differential pitches of sound. Auditory brain implants, or ABIs (also referred to as auditory brain stimulators) are used to treat a smaller number of recipients with bilateral degeneration of the auditory nerve. For such recipients, the auditory brain stimulator provides stimulation of the cochlear nucleus in the brainstem, typically with a planar electrode array; that is, an electrode array in which the electrode contacts are disposed on a two dimensional surface that can be positioned proximal to the brainstem. For ease of description, embodiments of the present invention are described herein primarily in connection with one type of electrically-stimulating hearing implant, a cochlear implant.

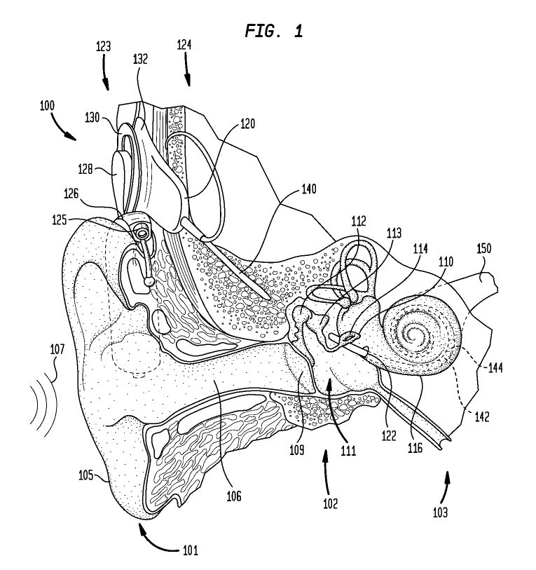

FIG. 1 is a perspective view of an exemplary cochlear implant 100 which may be configured for a recipient utilizing the programming techniques of the present invention. FIG. 1 is a cut-away view of the relevant components of outer ear 101, middle ear 102 and inner ear 103, which are described next below. In a fully functional ear, outer ear 101 comprises an auricle 105 and an ear canal 106. An acoustic pressure or sound wave 107 is collected by auricle 105 and channeled into and through ear canal 106. Disposed across the distal end of ear canal 106 is a tympanic membrane 109 which vibrates in response to acoustic wave 107. This vibration is coupled to oval window or fenestra ovalis 110 through three bones of middle ear 102, collectively referred to as the ossicles 111 and comprising the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 116. Such fluid motion, in turn, activates tiny hair cells (not shown) that line the inside of cochlea 116. Activation of the hair cells causes appropriate nerve impulses to be transferred through the spiral ganglion cells (not shown) and auditory nerve 150 to the brain (not shown), where they are perceived as sound. In deaf persons, there is an absence or destruction of the hair cells. Stimulator unit 120 is utilized to directly stimulate the ganglion cells to provide a hearing sensation to the recipient.

FIG. 1 also shows how a cochlear implant 100 is positioned in relation to outer ear 101, middle ear 102 and inner ear 103. Cochlear implant 100 comprises external component assembly 123 which is directly or indirectly attached to the body of the recipient, and an internal component assembly 124 which is temporarily or permanently implanted in the recipient. External assembly 123 comprises microphone 125 for detecting sound which is provided to a BTE (Behind-The-Ear) speech processing unit 126 that generates coded signals which are provided to an external transmitter unit 128, along with power from a power source such as a battery (not shown), and user controls (not shown). External transmitter unit 128 comprises an external coil 130 and, preferably, a magnet (also not shown) secured directly or indirectly in external coil 130. Internal assembly 124 comprises an internal receiver unit 132 having an internal coil (not shown) that receives and transmits power and coded signals from external assembly 123 to an implanted stimulator unit 120 to apply the coded signal along an electrode assembly 140. Electrode assembly 140 enters cochlea 116 at cochleostomy region 122 and has one or more electrodes 142 positioned to substantially be aligned with portions of cochlea 116.

Cochlea 116 is tonotopically mapped with each region of the cochlea being responsive to acoustic and/or stimulus signals in a particular frequency range. To accommodate this property of cochlea 116, stimulator unit 120 includes an array 144 of electrodes 142 each constructed and arranged to deliver appropriate stimulating signals to a particular region of the cochlea, each representing a different frequency component of a received audio signal. Signals generated by stimulator unit 120 are applied by electrodes 142 of electrode array 144 to cochlea 116, thereby stimulating auditory nerve 150. It should be appreciated that although in the embodiment shown in FIG. 1 electrodes 142 are arranged in an array 144, other electrode arrangements may be implemented.

In one example, electrode array 144 may include a plurality of independent electrodes 142 each of which may be independently stimulated. For example, in an embodiment employing Cochlear's Nucleus™ 24 system, electrode array 144 includes 22 independent electrodes each of which stimulates an area of auditory nerve 150 of the recipient's cochlea 116. As one of ordinary skill in the art is aware, low-frequency sounds stimulate the apical region of cochlea 116, while higher frequencies more strongly stimulate the basal region. Thus, one or more electrodes 142 of electrode array 144 located near the base of the cochlea are used to simulate high frequency sounds while electrodes closer to the apex are used to simulate lower frequency sounds. Because each electrode 142 or groups of electrodes 142 are used to stimulate a specific frequency or frequency band, these electrodes or groups of electrodes are sometimes referred to as a frequency channel, or simply channel, of the cochlear implant.

Furthermore, in many cochlear implants, speech processing unit 126 stimulates only the electrodes for which the stimulation signal has the greatest amplitude. For example, cochlear implant 100 may estimate the outputs for each of the 22 electrodes 142 and select the ones with the largest amplitude (that is, maxima). The number of maxima selected may vary, for example, between five and ten, depending on a variety of factors. Moreover, the rate of stimulation, often referred to in units of pulses per second, may also vary.

As one of ordinary skill in the art will appreciate, embodiments of the present invention may be used in combination with any speech strategy now or later developed including, but not limited to, Continuous Interleaved Sampling (CIS), Spectral PEAK Extraction (SPEAK), and Advanced Combination Encoders (ACE™). An example of such speech strategies is described in U.S. Pat. No. 5,271,397, the entire contents and disclosures of which is hereby incorporated by reference herein. Other examples also may also include front-end processing algorithms such as those described in U.S. Pat. No. 6,731,767 entitled ‘Adaptive dynamic range of optimization sound processor,’ WO 2005/006808 entitled ‘Method and Device for Noise Reduction.’ Moreover, a genetic algorithm may be used to optimize the MAP for features such as, but not limited to: rate, growth function and the like, as described in WO 2004/080532 entitled ‘Cochlear implant System with MAP Optimization Using a Genetic Algorithm.’ The above references are hereby incorporated by reference herein. The present invention may also be used with other speech coding strategies now or later developed. Certain embodiments of the present invention may be used in conjunction with Cochlear Limited's Nucleus™ implant system that uses a range of coding strategies alternatives, including SPEAK, ACE™, and CIS.

As noted, the operation of cochlear implant 100 is dependent, in part, on the success with which the implant is configured for an individual recipient. Fitting of the implant creates a recipient's MAP that defines the specific characteristics used to stimulate electrodes 142 of implanted electrode array 144. This MAP is sometimes referred to as the recipient's “program.”

Although a recipient's MAP may include a number of sophisticated parameters, the MAP typically includes at least two values for each channel of cochlear implant 100. These values are referred to as the Threshold level (commonly referred to as the “THR” or “T-level;” “threshold level” herein) and the Maximum Comfortable Loudness level (commonly referred to as the Most Comfortable Loudness level, “MCL,” “C-level,” simply “comfort level” herein). Threshold and comfort levels are psychophysical judgments of loudness that are measured in clinical units of electrical current, referred to as current units (cu). Threshold levels are comparable to acoustic threshold levels and indicate the current level at which a sound is barely audible. Comfort levels indicate the current level at which a sound is loud but comfortable.

Due to the currently common usage of threshold and current levels, exemplary embodiments of the present invention are described herein in the context of determining such MAP parameters for cochlear implant 100. As one of ordinary skill in the art would appreciate, however, the present invention may be used to perform fitting operations of the present invention for any MAP parameters of any electrically-stimulating hearing implant now known or later developed.

Advances in cochlear implant technology have resulted in a relatively complex fitting process during which a recipient's threshold and comfort levels are determined by a specially trained audiologist. These specially trained audiologists have a detailed understanding of how the electrical stimulation signals generated by the electrically-stimulating hearing implant are defined, generated and/or controlled. Audiologists lacking this knowledge are unable to perform the operations necessary to determine a recipient's threshold and comfort levels.

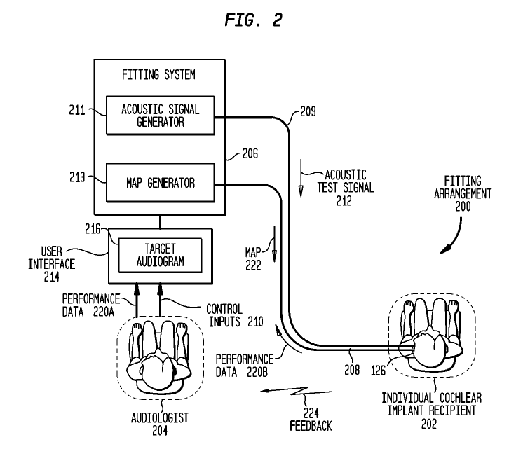

FIG. 2 is a schematic diagram of an exemplary fitting arrangement 200 in which embodiments of a hearing implant fitting system 206 is implemented to fit a cochlear implant for a recipient. As described below, fitting system 206 permits audiologists who lack detailed understanding of electrical stimulation to configure cochlear implant 100 (FIG. 1) for a recipient. Although FIG. 2 is discussed herein with reference to fitting cochlear implant 100, it should be appreciated that any electrically-stimulating hearing implant may be fit in the described manner.

As shown in FIG. 2, audiologist 204 uses a electrically-stimulating hearing implant fitting system 206 that comprises interactive software and computer hardware to configure an individualized recipient MAP 222 that is used for subsequent normal implant operations by speech processing unit 126 (FIG. 1). In addition to fitting cochlear implant 100 for recipient 202, fitting system 206 may be programmed and/or implements software programmed to carry out one or more of the functions of neural response measuring, acoustic stimulating, and/or recording of neural response measurements and other stimuli.

In accordance with the embodiments of FIG. 2, fitting system 206 includes an acoustic domain environment through which an audiologist 204 specifies for a variety of frequency channels the intensity level at which a particular recipient is to experience a desired percept. As described below, for each of these acoustic targets fitting system 206 determines the stimulating current level that evokes the desired percept in the recipient.

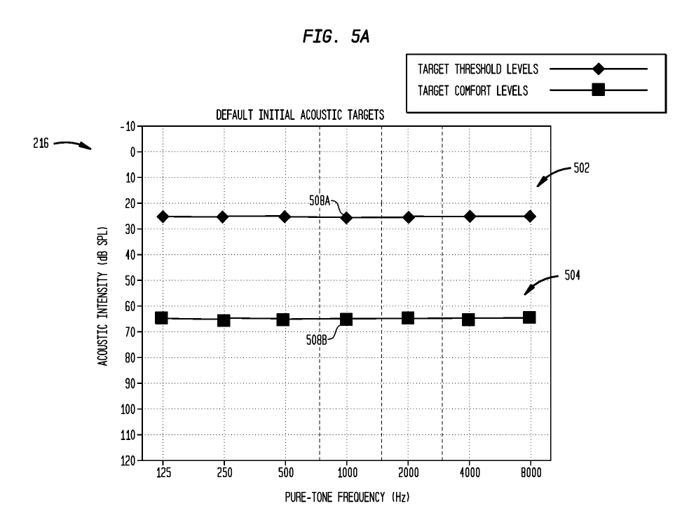

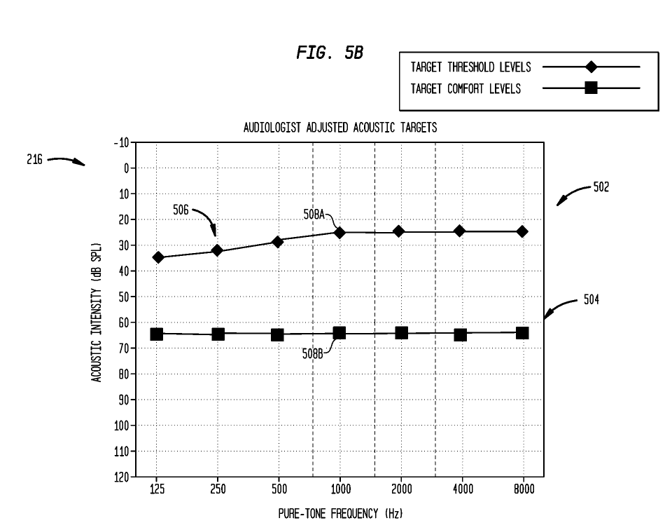

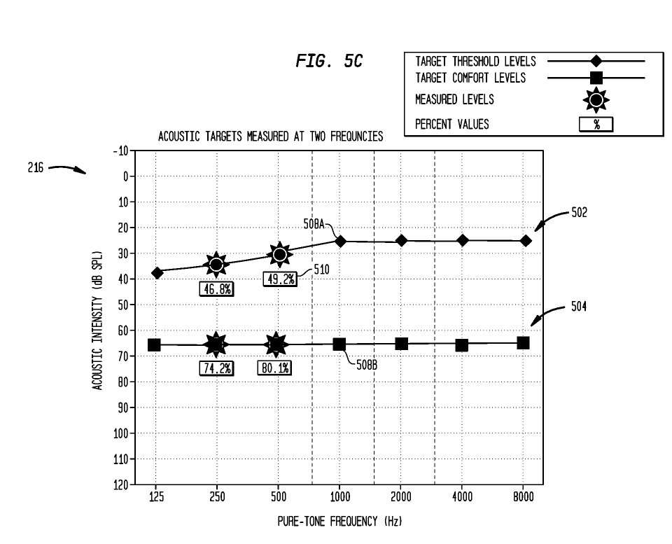

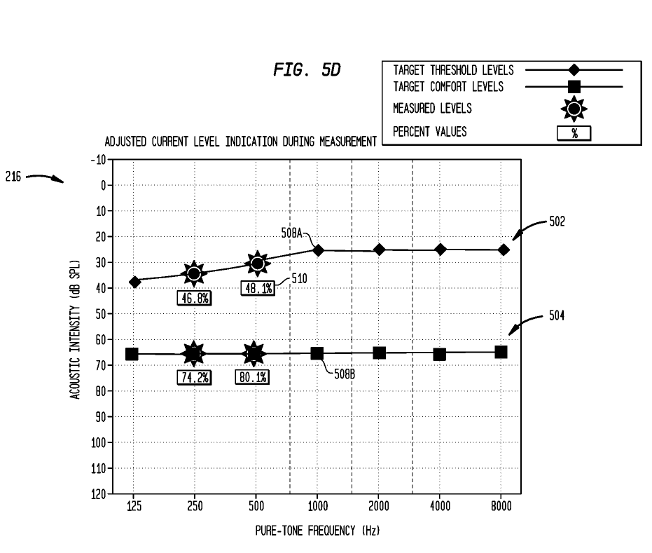

The acoustic domain environment presents fitting information or other data in the acoustic domain; that is, using acoustic-based data which is the form commonly used by audiologists to fit hearing aids. In embodiments of the present invention, the acoustic domain environment includes one or more graphs or plots 216 display on a user interface 214 that illustrate frequency-gain relationships, such as target audiogram 216 described in greater detail below with reference to FIGS. 5A-5D.

As noted, for each of the acoustic targets fitting system 206 determines the stimulating current level that evokes the desired percept in the recipient. Specifically, embodiments of fitting system 206 repeatedly present each acoustic target to the implant and increment or decrement the corresponding current level until recipient 202 experiences the desired percept in response to the acoustic target. These current levels are included in the MAP stored in the implant for subsequent normal implant operations by that recipient.

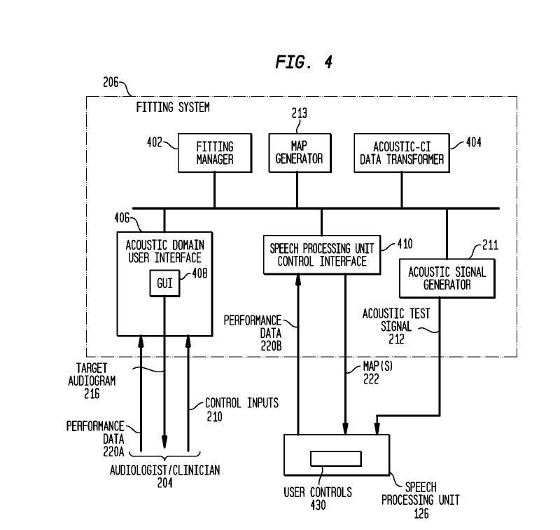

In embodiments of the present invention, an acoustic target is selected and MAP generator 213 generates a MAP 222. MAP 222 is loaded into the recipient's speech processing unit 126 (FIG. 1). As shown, MAP 222 may be loaded directly into speech processing unit 126 via programming cable 208. Although FIG. 2 illustrates a direct connection between MAP generator 222 and speech processing unit 126, it should be appreciated that fitting system 206 may comprise one or more components which interface the MAP generator with the speech processing unit. Such elements are shown in FIG. 4 as a speech processing control interface in FIG. 4. It should be appreciated that MAP 222 may be loaded into speech processing unit 126 in a variety of alternative manners, such as via wireless transmission.

After MAP 222 is loaded into speech processing unit 126, the selected acoustic target is presented to cochlear implant 100. The selected acoustic target may be presented as an acoustic test signal 212 having the intensity and frequency of the acoustic target. In certain embodiments, this acoustic test signal 212 may be generated by acoustic signal generator 211. As shown, acoustic test signal 212 may be presented to the recipient via audio cable 209. Alternatively, acoustic test signal 212 may be presented by free field transmission. In certain embodiments of the present invention, audiologist 204 presses a control button on user interface 214 to present acoustic test signal 212 to recipient 202.

Presentation of the selected acoustic target causes an electrical stimulation signal to be delivered to recipient at a current level corresponding to the selected acoustic target. The current level of the delivered stimulation signal is a default level corresponding to the selected target specified in MAP 222. The current level corresponding to the acoustic target specified is iteratively adjusted until presentation of the acoustic target evokes a desired percept.

More specifically, following presentation of the acoustic target, a determination is made as to whether the delivered stimulation signal evoked a desired percept. This determination is based on the response of recipient 202 to the stimulation. As shown in FIG. 2, the recipient's response may comprise verbal or non-verbal feedback 224 from recipient 202. In such embodiments, using feedback 224, audiologist 204 enters an indication at user interface 214 as to whether the desired percept was evoked. This indication is shown as performance data 220A. Audiologist 204 may enter performance data 220A using any one or combination of known methods, including a computer keyboard, mouse, voice-responsive software, touch-screen, retinal control, joystick, and any other data entry or data presentation formats now or later developed. Performance data 220A may simply indicate whether or not recipient 202 heard a sound following presentation of the acoustic target. Alternatively, functions may be provided which permit audiologist 204 to indicate if a perceived sound was too loud or too soft.

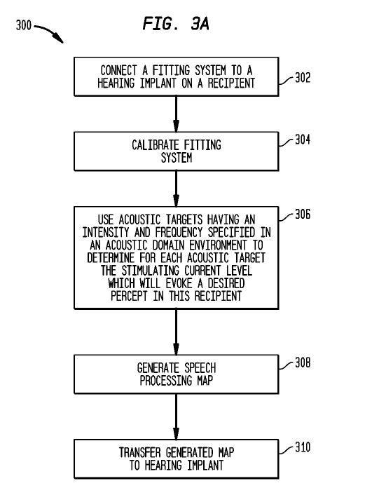

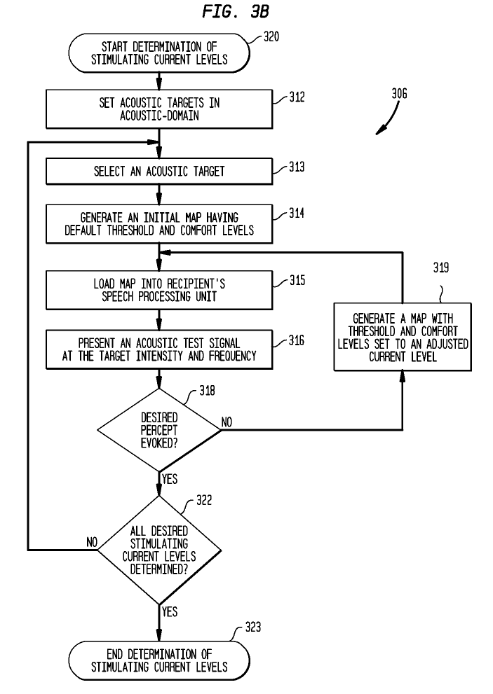

As described below in more detail with reference to FIG. 3B, if a desired percept is not evoked by presentation of the acoustic target, then the current level corresponding to the selected acoustic target is adjusted. More specifically, MAP generator 213 generates a new MAP 222 in which the current level of the relevant channel is increased or decreased, depending on the response of recipient 202. This new MAP 222 is loaded into speech processing unit 126 and the acoustic target is re-presented to recipient 202. Due to the adjusted current level specified in MAP 222, the response of recipient 202 will be different than the previous response. This process continues until presentation of acoustic target evokes a desired percept.

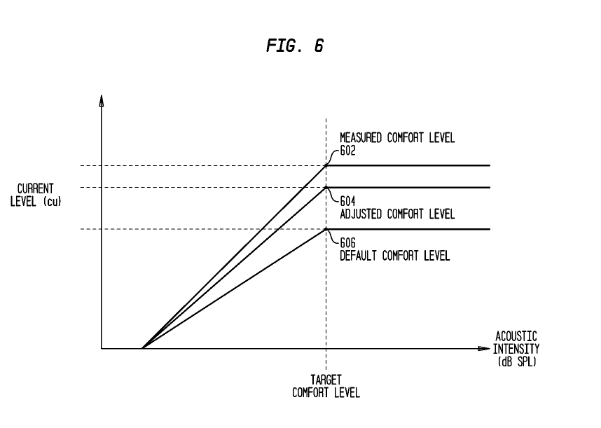

Determination that the desired percept was evoked indicates that the current level then specified in MAP 222 is the current level which will cause the desired percept at the target intensity and frequency. This value, referred to herein as the determined or measured current level, may be stored by fitting system 206 for later use in generating the recipient's MAP. The above process may be repeated until current levels corresponding to all acoustic targets have been determined. After determination of current levels corresponding to all desired acoustic targets, a MAP 222 is created and loaded into speech processing unit 126. As described in greater detail below with reference to FIG. 6, further adjustments may then be made to MAP 222 to meet the listening requirements of the recipient.

FIG. 3A is a high-level flow chart illustrating operations that may be performed while fitting an electrically-stimulating hearing implant on a recipient utilizing an embodiment of fitting system 206 (FIG. 2). In the illustrative embodiments, at block 302 the fitting system is bi-directionally coupled or connected to the electrically-stimulating hearing implant on the recipient. As described above with reference to FIG. 2, this connection may be a direct connection made by one or more wires or cables, such as an audio cable and a programming cable. In alternative embodiments, this connection may comprise a wireless connection.

Following connection, the fitting system is calibrated at block 304. Calibration ensures that an acoustic signal having a selected intensity is delivered to the electrically-stimulating hearing implant at the correct current level. This calibration may include the presentation of data as required by the implemented fitting protocol. Such calibration operations are well-known in the art and, therefore, are not described further herein.

Once the fitting system is calibrated, acoustic targets are used to determine, for each acoustic target, the stimulating current level which will evoke a desired percept in this recipient 306. The acoustic targets each have an intensity and frequency which are specified in an acoustic domain environment. The operations performed to determine or measure such current levels using acoustic targets are described in greater detail below with reference to FIG. 3B.

Following determination of all desired current levels, at block 308 a MAP for use in normal implant operations is generated using the measured current levels. The particular details of the implemented process for generating MAP 222 may be specific to the recipient, electrically-stimulating hearing implant manufacturer, electrically-stimulating hearing implant device, etc, and, as such, will not be described herein. However, it should be appreciated that it is not necessary to measure current levels, such as threshold and/or comfort levels, for each channel of the electrically-stimulating hearing implant. Rather, the generation of the MAP may rely upon interpolation to set non-measured threshold and comfort levels. At block 310 this MAP is downloaded to or otherwise transferred to the electrically-stimulating hearing implant. In some embodiments, different MAPs may be generated and stored for different listening situations.

In aspects of the present invention, following generation of the recipient's MAP, the audiologist may perform further adjustments to the measured current levels to meet the listening requirements of the recipient. These additional adjustments are discussed in greater detail below. Similarly, the recipient may also have the option to make various adjustments to the measured current levels.

FIG. 3B illustrates the operations performed for determining stimulating current levels using acoustic targets in accordance with embodiments of the present invention. The current level determination begins at block 320. At block 312, an audiologist sets acoustic targets by specifying the intensity level at which a particular recipient is to experience a desired percept for a variety of frequency channels. As noted above, an acoustic domain environment is provided through which the audiologist sets the acoustic targets. In some embodiments, each of the acoustic targets corresponds to a threshold and/or comfort level to be measured.

Following setting of the acoustic targets, at block 313 an acoustic target is selected for determination of the current level corresponding thereto. At block 314, an initial MAP is generated. In this MAP, the threshold and comfort levels of the channel corresponding to the frequency of the selected acoustic target (referred to as the relevant channel herein) are both set to a default current level. The default current levels may be arbitrary, but would generally be higher or lower depending if the acoustic target corresponds to a threshold or comfort level. The threshold and comfort levels of the remaining channels are set to a minimum current level, such as 0 current units (cu). This generated MAP is loaded into the recipient's speech processing unit at block 315. As noted above, the MAP may be transferred or loaded into speech processing unit via a programming cable 208, wireless connection, etc.

As noted, in embodiments of the present invention, the threshold and comfort levels for the relevant channel are both set to the same current level. These two different levels are both set to the same current level because during each measurement of a current level corresponding to an acoustic target, there is only one current level at which stimulation is delivered to the recipient. As such, there is no need to have independent threshold and current levels. The linking of the threshold and comfort levels may simplify the generation of the initial MAP at block 314. It should be appreciated that it alternative embodiments, it is not necessary to set threshold and comfort levels to the same level. Similarly, in alternative embodiments, it is not necessary to set the current levels of remaining channels to a minimum current level. These alternative embodiments will not be described herein, but are within the scope of the present invention.

At block 316, the selected acoustic target is presented to the recipient. In the illustrative embodiment, the acoustic target is presented as an acoustic test signal having the intensity and frequency of the selected target via audio cable 209. The electrically-stimulating hearing implant delivers the acoustic test signal via the relevant channel as an electrical stimulation signal. The stimulation signal is delivered at the above default current level.

A determination is then made at block 318 as to whether the acoustic test signal evoked a desired or expected percept. This determination may be made based on the recipient's response to the delivered stimulation signal. As noted above, the recipient's response to the stimulation signal may comprise verbal or non-verbal feedback from the recipient. In the illustrative embodiments, a desired percept occurs when the acoustic test signal is loud but comfortable (comfort level) or when the acoustic test signal is barely audible (threshold level). If the recipient's response indicates that the acoustic test signal did not evoke a desired percept, a new MAP is generated at block 319. In this MAP, the current level of the relevant electrode increased or decreased, depending on the recipient's response and the selected target. For example, when measuring a threshold level, if the acoustic test signal is inaudible, the current level of the relevant electrode would be increased. As would be appreciated, similar appropriate adjustments would be made following different responses and with different selected targets.

The newly generated MAP having the adjusted current level on the relevant channel is loaded into the recipient's speech processing unit at block 315. The acoustic test signal is re-presented to the recipient at the target intensity and frequency to determine if a desired percept is evoked. The current level of the relevant channel would be incrementally adjusted in the above manner until the desired percept is evoked.

Once the desired percept is evoked for the selected acoustic target, another determination is made at block 322 as to whether all desired currents levels have been determined. If all desired levels have not been determined, the above process may be repeated for all acoustic targets specified by the audiologist. As described above, after all desired acoustic targets have been selected, the current level determination ends and the recipient's MAP is generated as shown in FIG. 3A.

FIG. 4 is a high-level functional block diagram of electrically-stimulating hearing implant fitting system 206 according to one embodiment of the present invention. The primary components and operative aspects of fitting system 206 are shown in block diagram form for ease of description, and are described herein. The primary components are interoperably coupled to perform fitting operations. In the exemplary embodiment shown in FIG. 4, the components are shown as being coupled by a communications bus. However, it is to be understood that the components of fitting system 206 may be operably coupled in any manner suitable for the particular application.

Fitting manager 402 performs fitting operations and controls the other components shown in FIG. 4. The operation and other aspects of fitting manager 402 are known in the art and are not described further herein.

As described above, fitting system 206 provides an acoustic domain environment through which an audiologist specifies for a variety of frequency channels the intensity level at which a particular recipient 202 is to experience a desired percept. For each of these acoustic targets fitting system 206 determines the stimulating current level that evokes the desired percept in recipient 202. Specifically, embodiments of fitting system 206 repeatedly present each acoustic target to the implant and increment or decrement the corresponding current level until recipient 202 experiences the desired percept in response to the acoustic target.

As noted, each acoustic target may be presented to the recipient as an acoustic test signal, shown as test signal 212. Acoustic signal generator 211 generates acoustic test signal 212 as noted above. Acoustic test signal 212 can be sent to recipient 202 via a channeling, directing or other intermediate device, such as an audio cable 209 as shown in FIG. 2. In FIG. 4, for example, acoustic test signal 212 is provided electronically to speech processing unit 126. In other embodiments, acoustic test signal 212 can be sent by free field transmission. Free field transmission refers to sending acoustic signals through air to recipient 202. It is to be understood that acoustic test signal 212 can be transmitted by acoustic signal generator 211 through the aforementioned techniques, as well as any techniques or devices now or later developed. Alternatively, acoustic signal generator 211 could be omitted from fitting system 206. In these embodiments, fitting system 206 would generate a set of instructions based on the selected acoustic target, and these instructions would be executed by speech processing unit 126 to stimulate the recipient.

Fitting system 206 is operably coupled to cochlear implant 100. In the embodiment shown in FIG. 4 fitting system 206 includes a speech processor control interface 410 to provide such interoperability. Performance data 220B may be sent from cochlear implant 100 to fitting system 206. As described below, this performance data 220B may include indications of the current level delivered to a recipient during the above described measurement. Also, data such as MAP(s) 222 generated by MAP generator 213 may be sent to cochlear implant 100 through speech processor interface 410.

User interface 406 may include any interface which is used by audiologist 204 to communicate with fitting implant system 206. Audiologist 204 can provide input using any one or combination of known methods, including a computer keyboard, mouse, voice-responsive software, touch-screen, retinal control, joystick, and any other data entry or data presentation formats now or later developed.

In the embodiment illustrated in FIG. 4, user interface 406 includes a graphical user interface (GUI) 408 which is displayed on user interface 406, as noted above. As noted, user interface 406 presents and receives fitting information in the acoustic domain. In this exemplary embodiment, such information is presented as target audiogram 216.

In certain embodiments of the present invention, recipient 202 has the option to make various adjustments to the current levels MAP 222 following the fitting procedure described above. For example, the recipient may adjust the measured current levels so that the overall volume is increased or decreased. Similarly, recipient 202 may make incremental adjustments to the low, mid, and high tone settings. In certain embodiments, these adjustments may provide equivalent adjustments which are available in the fitting software.

In specific such embodiments, the volume control will operate, for example, in a pre-set range, +/−10% of the dynamic range between threshold and comfort levels. Similarly, pre-set ranges for tone adjustment may also be available. These pre-set percents are programmable by the audiologist. As shown in FIG. 4, speech processing unit 126 may comprise user controls 430 that permit such adjustment. Alternatively, such controls may also be provided, for example, on a remote control which communicates with the speech processing unit using radio frequencies. In certain embodiments, user controls 430 may include a visual display of tone and volume settings and one or more controls which permit the above described adjustment of the tone/volume.

As noted above, fitting system 206 provides an acoustic domain environment through which audiologist 204 specifies for a variety of frequency channels the intensity level at which a particular recipient is to experience a desired percept. In certain embodiments, the acoustic domain environment includes a target audiogram 216 to permit audiologist 204 to set such acoustic targets. FIGS. 5A-5D illustrate exemplary target audiograms 216 which may be displayed to audiologist 204 in embodiments of the present invention.

Target audiogram 216 illustrated in FIGS. 5A-5D fitting information or other data in the acoustic domain; that is, using acoustic-based data which is the form commonly used by audiologists to fit hearing aids. In embodiments, each acoustic target 508 is displayed in target audiogram 216 at an acoustic signal intensity and frequency. In accordance with embodiments of the present invention, any measure or representation of intensity, such as Watts/square meter, decibels, etc, may used in target audiogram 216. In specific embodiments of the present invention, the intensities of acoustic targets 508 are specified in dB SPL; that is, dB with referenced to a fixed level, or dB HL; that is, dB referenced to normal hearing, depending on the preferences of audiologist 204. As such, fitting system 206 may have the ability to provide target audiograms in either dB HL or dB SPL or in any other measure or representation of intensity.

As shown in FIG. 5A, in certain embodiments, at the commencement of the fitting procedure target audiogram 216 includes a plurality of acoustic targets 608 set at default acoustic intensities and frequencies. These default acoustic intensities may be, for example, ideal or preferred acoustic intensities at which it is desirable for a recipient to hear a sound. For example, fitting system 206 may initially display 25 dB for target thresholds and 65 dB for target comforts. In the illustrative embodiments, target audiogram 216 comprises seven pure-tone octave frequencies at which acoustic targets 508 are set by audiologist 204. It should be appreciated that more or less frequencies may also be used. As shown, the audiologist may set multiple acoustic targets 508 for each frequency. For example, the audiologist may set target thresholds 502 and target comforts 504 for each frequency.

As noted, target audiogram 216 is displayed using seven pure-tone frequencies. Alternatively, rather than using pure-tone frequencies, narrow bands of noise or filtered speech signals could be used. This could increase the number of electrodes stimulated with each acoustic test signal and could reduce programming time because less threshold and/or comfort levels would need to be measured. This may also reduce the need for further adjustment because perceptual integration from multiple electrode stimulation has been captured.

Audiologist 204 may adjust the acoustic intensity levels of acoustic targets 208 from the default levels depending on the listening needs of the recipient, typical usage environment, etc. These adjustments may be made using, for example, control inputs 210 described above. As shown in FIG. 5B, when audiologist adjusts the intensities, the symbols representing the acoustic target move up or down on the screen. In the illustrative embodiment of FIG. 5B, audiologist 204 has adjusted target threshold levels 508 in the lower frequency range by increasing the acoustic intensity thereof. This is shown by the movement of the symbols in lower frequency range 506 in FIG. 5B. An increase in the intensity of target threshold levels in lower frequency 506 would be desirable, for example, if the recipient's typical use environment includes low-intensity and low-frequency background noise.

As described above, fitting system 206 measures the current levels corresponding to each acoustic target 508 that will result in a desired percept when an acoustic signal is presented to cochlear implant 100 at the target frequency and intensity. In some embodiments, indications of these measured current levels may optionally be provided from speech processing unit 126 to fitting system 206 for display to audiologist 204. These indications may be provided from speech processing unit 126 as performance data 220B shown in FIGS. 2 and 4.

Rather than displaying the measured current levels directly to audiologist 204, fitting system 206 may display a representation of the measured current in a user friendly format which does not require an understanding of the current levels. In such embodiments, target audiogram 216 represents the measured current as a percentage of the total current available to stimulate the recipient. For example, in certain cochlear implants, current levels from 0-255 current level units (cu) are available for electrical stimulation of recipient 202. Thus, in such an example, the measured current would be expressed as a percentage of 255 cu. The percent values 510 shown in FIG. 5C provide the audiologist with clinical guidance about relative differences in the required current levels across the tested frequencies.

In some embodiments, the indications of the measured current level are updated and displayed to audiologist 204 after each adjustment of the current level in the recipient's MAP as described above. Such changes are illustrated in FIG. 5D with reference to percent value 510. In this illustrative embodiment, the current level corresponding to the acoustic threshold at 500 Hz is undergoing measurement. As shown, the percent value 510 decreased from 49.2% to 48.1%. This change indicates that the previous current delivered to the recipient result in a percept that was above the recipient's threshold level. Displayed percent value 510 may be continually updated until the desired percept is evoked, at which time the percent value would indicate the measured level.

It should appreciated that target audiogram 216 may be displayed on any suitable apparatus or media. Similarly, although embodiments of the present invention are discussed herein with reference to target audiogram 216, other presentations of frequency-gain relationships are feasible. For example, it should appreciated that such frequency-gain relationships may be represented in other forms including, but not limited to, bar graphs, tables, histograms, drop-down menus, radio buttons, text boxes, or any other method for displaying such data.

As noted above, in certain embodiments of the present invention, following measurement of the threshold and/or comfort levels, audiologist 204 may further adjust the current levels using fitting system 206. In certain embodiments, this adjustment may be performed using a voice-over procedure. During such a voice-over procedure, audiologist 204 speaks with recipient 202 so that further adjustment of overall volume or adjustments at different frequency regions can be made. Alternatively, audiologist 204 may play pre-recorded speech to recipient 202.

When audiologist 204 speaks with recipient 202 during the voice-over procedure, the recipient may indicate that the overall volume is too soft or too loud. In such embodiments, audiologist 204, using one or more control inputs 210, would make adjustments to the measured comfort levels across all frequencies to increase or decrease the overall volume. In other embodiments, when audiologist 204 speaks with recipient 202, the recipient indicates that one or more of the high, mid or low frequency ranges are too soft or too load. As such, audiologist 204 adjusts the comfort levels of certain frequency ranges (bass/midrange/treble boost or cut). Threshold levels could further be adjusted by the audiologist in substantially the same manner.

As just noted, audiologist 204 may make adjustments to overall volume or to particular frequency ranges. Alternatively, as discussed above with reference to FIG. 4, speech processing unit 126 may include one or more controls 430 that permit the recipient or other user, such as audiologist 204, to make similar adjustments to overall volume or to particular frequency ranges. The adjustments provided by controls 430 on speech processing unit 126 would be substantially the same as those provided to audiologist 204 at fitting system 206. In operation, either of the above adjustments directly change the current level of a channel of cochlear implant 100. Such a change is illustrated in FIG. 6 with reference to a single comfort level.

A default comfort level 606 for a single channel is shown in FIG. 6. Using the fitting procedure, the comfort level for the channel was measured as current level 602. After measurement, a determination is made that this comfort level should be decreased. This determination may be made in any of the manners described above. As a result, using either controls 430 provided on speech processing unit 126 or control inputs 210 of fitting system 206, the comfort level is decreased in the recipient's MAP to adjusted current level 604. As noted, these adjustments to the recipient's MAP may occur at each frequency, a range of frequencies, etc.

As noted above, a recipient's MAP may include various parameters. The most widely used MAP parameters include the recipient's threshold and comfort levels because these levels are limits of audibility and comfort, respectively. As such, for ease of description, embodiments of the present invention have been described herein with reference to measurement of these widely used threshold and comfort levels. It should be appreciated that any other levels or MAP parameters may be measured in accordance with embodiments of the present invention.

Furthermore, embodiments of the present invention have been primarily described with reference to the use of verbal or non-verbal feedback to determine if a desired percept has been evoked. It should be appreciated that in alternative embodiments, objective measurements, such as neural response measurements, obtained before, during or after delivery of the electrical stimulus may be used to determine if a desired percept has been evoked.

As noted above, in embodiments of the present invention both threshold and comfort levels are measured at a target frequency. In alternative embodiments of the present invention, one of either threshold or comfort levels would be measured at each target frequency and the other level at each frequency would be estimated based on the measured value. In these embodiments, selected individual estimated levels may be adjusted using any of the methods described above. Embodiments of the present invention may use a combination of these estimation embodiments and the full measurement embodiments described above. For example, threshold and comfort levels could both be measured at several frequencies and one or more of the threshold or comfort levels could be estimated at other frequencies.

Further features of the present invention are described in U.S. patent application Ser. No. 11/348,309, filed Feb. 2, 2007 and U.S. Provisional Application No. 60/650,148, filed Feb. 7, 2005, both of which are hereby incorporated by reference herein in their entirety. Other information may be found in (1) Gitte Keidser, et al. “Using the NAL-NL1 prescriptive procedure with advanced hearing aids.” National Acoustic Laboratories. Mar. 2, 2006, pages 1-10. (Reprinted with permission from The Hearing Review, November 1999.); and (2) Teresa Y. C. Ching, et al. “RECD, REAG, NAL-NL1: Accurate and practical methods for fitting non-linear hearing aids to infants and children.” National Acoustic Laboratories, Reprinted with permission from The Hearing Review, August 2002, vol. 9, no. 8, pages 12-20, 52.

All documents, patents, journal articles and other materials cited in the present application are hereby incorporated by reference herein.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. A method for fitting a cochlear implant for a recipient, comprising:
receiving, at an acoustic domain user interface, an acoustic target intensity for each of a plurality of frequency channels at which the recipient is to experience a desired percept;
loading into a speech processor unit of the cochlear implant a MAP specifying a stimulation signal current level corresponding to a selected acoustic target;
presenting said selected acoustic target to the cochlear implant so as to cause the cochlear implant to deliver electrical stimulation to the recipient at said current level corresponding to said selected acoustic target; and
upon receipt of an external command:
adjusting said current level corresponding to said selected acoustic target; and
repeating said loading and said presenting steps.

2. The method of claim 1, wherein said current level corresponding to said selected acoustic target which evokes said desired percept is included in a MAP stored in the cochlear implant for subsequent normal implant operations

3. The method of claim 1, wherein said adjusting said current level comprises:
adjusting said current level based on the recipient's response to said stimulation.

4. The method of claim 1, further comprising:
presenting each acoustic target received at said user interface to the cochlear implant to determine the current level corresponding to each acoustic target which evokes a desired percept in the recipient.

5. The method of claim 1, wherein said acoustic domain user interface displays a target audiogram to an audiologist, and wherein said method further comprises:
setting said acoustic signal intensities in said target audiogram.

6. The method of claim 1, wherein said intensities of said acoustic targets are specified in one or more of dB hearing level (dB HL) and dB sound pressure level (dB SPL).

7. The method of claim 1, wherein said acoustic targets each correspond to one of a recipient's threshold and comfort levels.

8. The method of claim 1, further comprising:
displaying an indication of said current level corresponding to said selected acoustic target via said acoustic domain user interface.

9. The method of claim 1, further comprising:
performing a voice-over procedure to further adjust in said MAP said current level corresponding to said selected acoustic target.

10. The method of claim 1, wherein the cochlear implant comprises user controls, and wherein said method further comprises:
adjusting, with said user controls, said current level in said MAP corresponding to said selected acoustic target.

11. A system for fitting a cochlear implant for a recipient, comprising:
means for receiving, at an acoustic domain user interface, an acoustic target intensity for each of a plurality of frequency channels at which the recipient is to experience a desired percept;
means for loading into a speech processor unit of the cochlear implant a MAP specifying a stimulation signal current level corresponding to a selected acoustic target;
means for presenting said selected acoustic target to the cochlear implant so as to cause the cochlear implant to deliver electrical stimulation to the recipient at said current level corresponding to said selected acoustic target; and
upon receipt of an external command:
means for adjusting said current level corresponding to said selected acoustic target; and
means for repeating said loading and said presenting steps.

12. The system of claim 11, wherein said current level corresponding to said selected acoustic target which evokes said desired response is included in a MAP stored in the cochlear implant for subsequent normal implant operations

13. The system of claim 11, wherein said current level is adjusted based on the recipient's response to said stimulation.

14. The system of claim 11, further comprising:
means for presenting each acoustic target received at said user interface to the cochlear implant to determine the current level corresponding to each acoustic target which evokes a desired percept in the recipient.

15. The system of claim 11, wherein said means for receiving said acoustic targets comprises a target audiogram.

16. The system of claim 11, wherein said intensities are specified in one or more of dB hearing level (dB HL) and dB sound pressure level (dB SPL).

17. The system of claim 11, wherein each said acoustic target corresponds to one of a recipient's threshold and comfort levels.

18. The system of claim 11, further comprising:
means for displaying an indication of said current level corresponding to said selected acoustic target via said acoustic domain user interface.

19. The system of claim 11, further comprising:
means for performing a voice-over procedure to further adjust in said MAP said current level corresponding to said selected acoustic target.

20. The system of claim 11, wherein the cochlear implant comprises:
means for adjusting in said MAP said current level which evokes said desired percept.

21. A system for fitting a cochlear implant for a recipient, comprising:
an acoustic domain user interface configured to receive an acoustic target intensity for each of a plurality of frequency channels at which the recipient is to experience a desired percept;
a MAP generator configured to load a MAP into a speech processor unit of the cochlear implant, said MAP specifying a stimulation signal current level corresponding to a selected acoustic target;
an acoustic signal generator configured to present the selected acoustic target to the cochlear implant so as to cause the cochlear implant to deliver electrical stimulation to the recipient at said current level corresponding to said selected acoustic target; and
upon receipt of an external command, said system is configured to adjust said current level corresponding to said selected acoustic target, said MAP generator is configured to repeat said loading, and said signal generator is configured to repeat said presentation.

22. The system of claim 21, wherein said current level corresponding to said selected acoustic target which evokes said desired response is included in a MAP stored in the cochlear implant for subsequent normal implant operations

23. The system of claim 21, wherein said current level corresponding to said selected acoustic target is adjusted based on the recipient's response to said stimulation.

24. The system of claim 21, wherein each acoustic target received at said user interface is presented to the cochlear implant to determine the current level corresponding to each acoustic target which evokes a desired percept in the recipient.

25. The system of claim 21, wherein said acoustic domain user interface is configured to display a target audiogram to an audiologist, and wherein said audiologist sets said acoustic intensities in said target audiogram.

26. The system of claim 21, wherein said intensities are specified in one or more of dB hearing level (dB HL) and dB sound pressure level (dB SPL).

27. The system of claim 21, wherein said acoustic targets each correspond to one of a recipient's threshold and comfort levels.

28. The system of claim 21, wherein said acoustic domain user interface is configured to display an indication of said current level corresponding to said selected acoustic target.

29. The system of claim 21, wherein said indication of said current level comprises:
a ratio of the magnitude of said delivered current level to the total current available for delivery of electrical stimulation to the recipient.

30. The system of claim 21, wherein the cochlear implant comprises user controls permitting at least one of an audiologist and the recipient to further adjust in said MAP said current level corresponding to said acoustic target.

31. A method for fitting for a recipient a cochlear implant using acoustic targets each having an acoustic intensity and frequency specified in an acoustic domain environment, comprising:
determining for each acoustic target the stimulating current level which will evoke a desired percept in the recipient.

32. The method of claim 31, wherein determining the stimulating current level for a selected acoustic target which will evoke a desired percept in the recipient comprises:
loading into a speech processor unit of the cochlear implant a MAP specifying a stimulation signal current level corresponding to said selected acoustic target;
presenting said selected acoustic target to the cochlear implant so as to cause the cochlear implant to deliver electrical stimulation to the recipient at said current level corresponding to said selected acoustic target; and
upon receipt of an external command:
adjusting said current level corresponding to said selected acoustic target; and
repeating said loading and said presenting steps.

33. The method of claim 31, wherein said stimulating current levels are included in a MAP stored in the cochlear implant for subsequent normal implant operations

34. The method of claim 32, wherein said adjusting said current level corresponding to said selected acoustic target comprises:
adjusting said current level based on the recipient's response to said stimulation.

35. The method of claim 31, wherein an audiologist enters said acoustic targets at an acoustic domain user interface.

36. The method of claim 35, wherein said acoustic domain user interface displays a target audiogram to an audiologist, and wherein said method further comprises:
setting said acoustic signal intensities in said target audiogram.

37. The method of claim 31, wherein said intensities of said acoustic targets are specified in one or more of dB hearing level (dB HL) and dB sound pressure level (dB SPL).

38. The method of claim 31, wherein said acoustic targets each correspond to one of a recipient's threshold and comfort levels.
